(19) United States
(12) Patent Application Publication
(10) Pub. No.: US 2006/0155356 A1
(43) Pub. Date: Jul. 13, 2006

(54) Balloon catheter

(76) Inventor: Henry M. Israel, Bnei Brak (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 11/033,749

(22) Filed: Jan. 13, 2005

(51) Int. Cl. A61F 2/06

(52) U.S. Cl. 623/1.11

(57) ABSTRACT

A catheter including at least one inflatable chamber disposed on a catheter stem and in fluid communication with at least one inflation lumen in the catheter stem, the at least one inflatable chamber including an expandable covering with an underfold fluidly sealed to the catheter stem, the underfold including a portion of the expandable covering that is folded underneath itself.

### FIELD OF THE INVENTION

The present invention relates generally to stent catheters, and particularly to a catheter with one or more inflatable balloons that can be used for implanting bifurcated stents in body lumens.

### BACKGROUND OF THE INVENTION

There are bifurcated lumens, such as but not limited to, the carotid artery, which may need support with a bifurcated stent in procedures, such as but not limited to, percutaneous transluminal coronary angioplasty (PTCA). A bifurcated lumen (also called bifurcation) is an area of the vasculature where a first vessel is bifurcated into two or more branch vessels. Stenotic lesions may form in or around such bifurcations, that is, in or around one or more of the vessels.

An example of a prior art balloon catheter for introducing bifurcated stents is shown in FIG. 1 and is based on U.S. Pat. No. 6,494,905 to Zedler et al. A balloon catheter 1 includes a catheter stem 2 with a balloon that comprises two inflatable chambers 1.1 and 1.2, which are longitudinally spaced from each other along the length of the balloon catheter 1. The inflatable chambers 1.1 and 1.2 can either be filled separately or simultaneously by way of filling openings 3.1 and 3.2 via at least one feed passage (not shown) disposed within the stem 2 of the catheter 1.

A first guide wire 4 is disposed within the catheter stem 2 and exits the stem 2 laterally from a lateral exit opening 5 on the catheter stem 2 between the two inflatable chambers 1.1 and 1.2 of the balloon catheter 1. A second guide wire 6 is disposed within the catheter stem 2 and can be extended from the catheter stem 2 through a distal exit opening 7 to provide longitudinal guidance to the balloon catheter 1.

A problem of this and other prior art catheters for bifurcated stents is that the inflatable chambers must be sealed by seals 8 on the outside surface of catheter stem 2. A space must be provided on the catheter stem 2 between the seals 8 of adjacent inflatable chambers for the lateral exit opening 5. The purpose of the inflatable chambers is to inflate and cause expansion of a stem mounted on the catheter 1. However, because of the space between the seals 8, the adjacent inflatable chambers cannot support or cause expansion of anything surrounding that space between the seals 8. As a result, there is a zone of non-expansion between the balloons of prior art catheters.

### SUMMARY OF THE INVENTION

The present invention seeks to provide a catheter with one or more inflatable balloons that can be used for implanting bifurcated stents in body lumens. Contrary to the prior art, in the present invention, there is no zone of non-expansion between the balloons, as is described further hereinbelow.

There is provided in accordance with an embodiment of the present invention a catheter including at least one inflatable chamber disposed on a catheter stem and in fluid communication with at least one inflation lumen in the catheter stem, the at least one inflatable chamber including an expandable covering with an underfold fluidly sealed to the catheter stem, the underfold including a portion of the expandable covering that is folded underneath itself. There may be two or more inflatable chambers disposed longitudinally along the catheter stem. The inflatable chambers may abut one another in an inflated state.

In accordance with an embodiment of the present invention, the catheter stem may include one or more lateral exits disposed between the inflatable chambers. Another inflatable chamber may be disposed on the catheter stem. The inflatable chambers may share a common seal that fluidly seals them to the catheter stem.

A first guide wire may be disposed within the catheter stem. A second guide wire may be adapted to exit the catheter stem through the one or more lateral exits.

A deformable stent may be positioned coaxially around the inflatable chambers and adapted to radially deform upon application of an expansive force. The deformable stent may include a deformable side aperture.

### BRIEF DESCRIPTION OF DRAWINGS

The present invention will be further understood and appreciated from the following detailed description taken in conjunction with the drawing in which:

FIG. 1 is a simplified pictorial illustration of a prior art catheter with multiple balloons;

FIG. 2 is a simplified illustration of a catheter with inflatable chambers, constructed and operative in accordance with an embodiment of the invention;

FIG. 3 is a simplified illustration of a catheter with inflatable chambers, constructed and operative in accordance with another embodiment of the invention; and

FIG. 4 is a simplified illustration of a catheter with inflatable chambers, constructed and operative in accordance with yet another embodiment of the invention.

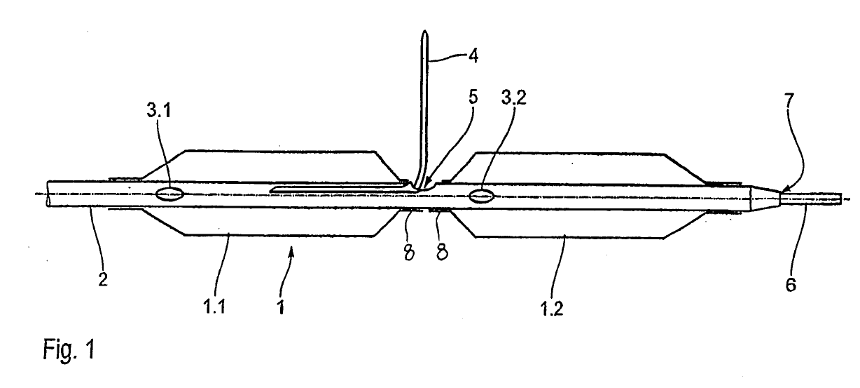

### DETAILED DESCRIPTION OF EMBODIMENTS

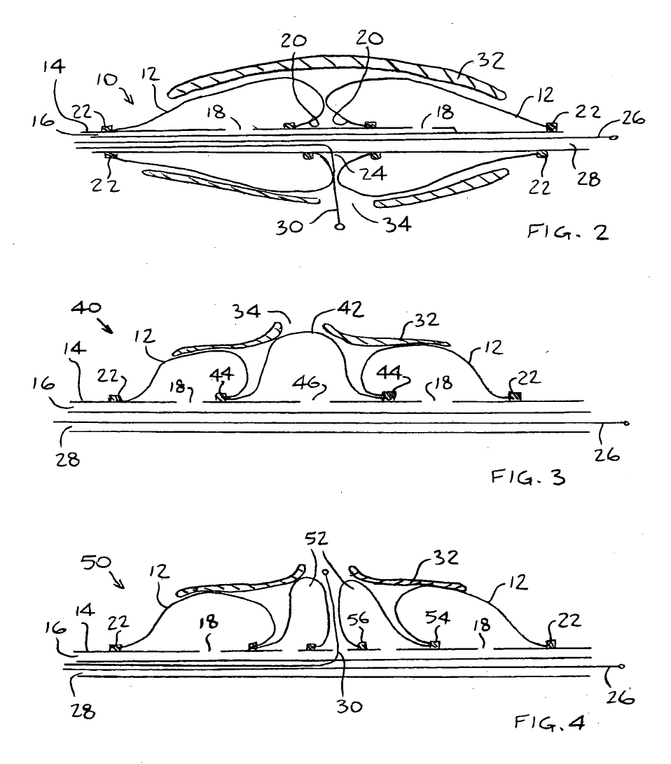

Reference is now made to FIG. 2, which illustrates a catheter 10, constructed and operative in accordance with an embodiment of the invention.

Catheter 10 may be constructed of any suitable catheter material, such as but not limited to, silicone rubber (e.g., SILASTIC from Dow Corning), polyurethane, polyethylene and the like. Catheter 10 may include one or more inflatable chambers 12 (also referred to as balloons 12) disposed on a catheter stem 14. Balloons 12 are in fluid communication with one or more inflation lumens 16 in catheter stem 14 via inflation ports 18.

The inflatable chamber 12 is constructed of an expandable covering with an underfold 20 fluidly sealed to catheter stem 14 by means of a seal 22. The underfold 20 includes a portion of the expandable covering that is folded underneath itself.

The expandable covering of balloons 12 may be any suitable inflatable material used for balloon angioplasty, such as but not limited to, PET (polyethylene terephthalate), polyethylene, polyvinyl chloride, SURLYN polyethylene ionomer copolymer, nylon 12, PEBAX polyamide-polyether-polyester block copolymer and others. The balloon material may be selected to have any degree of compliance, depending on the application. “Compliance” refers to how a material deforms (e.g., stretches) under pressure. Deformation of the material under pressure prevents forces from being concentrated at the site of the stenosis. The less compliant the balloon material, the more dilating or expanding force it transmits.

Seal 22 may be formed in any manner that produces a fluid-tight or fluid-proof seal to prevent leakage of fluids therepast (to any degree of sealing tightness, depending upon the application), and may be formed, without limitation, by sonic welding, bonding, for example, or any other suitable method of joining or sealing materials together.

In the non-limiting illustrated embodiment, there are two or more inflatable chambers 12, which are disposed longitudinally along catheter stem 14. Unlike the prior art, the inflatable chambers 12 may abut one another in the inflated state.

The catheter stem 14 may include one or more lateral exits 24 disposed between the inflatable chambers 12. A first guide wire 26 may be disposed within a guidewire lumen 28 in catheter stem 14. A second guide wire 30 may be disposed within guidewire lumen 28, and may be manipulated to exit catheter stem 14 through lateral exit 24.

A deformable stent 32 may be positioned coaxially around inflatable chambers 12 and adapted to radially deform upon application of an expansive force, as is well known in the art. For example, stent 32 may be constructed from a suitable balloon-expandable material, such as but not limited to, stainless steel 316L. Stent 32 may be a coated stent, such as a drug-eluting stent that has a polymer coating that emits an anti-restenosis drug. Stent 32 may be formed with a deformable side aperture 34 for placing a branch stent (not shown) at a site of a bifurcation.

As is well known in the art, catheter 10 may be passed over first guide wire 26 to enter a body lumen (not shown), such as but not limited to, a blood vessel. The first guide wire 26 may be introduced by means of an introducer sheath (not shown) inserted through some lumen (e.g., femoral artery), wherein the first guidewire 26 is manipulated through vasculature to the site of implanting the stent 32. The guidewires 26 and 30 may be grasped and manipulated (e.g., pushed, pulled, twirled and twisted) at their proximal ends, and may be constructed of any suitable material for guidewires, as is well known in the art.

Reference is now made to FIG. 3, which illustrates a catheter 40, constructed and operative in accordance with another embodiment of the invention. Catheter 40 is similar to catheter 10, with like elements being designated by like numerals.

Catheter 40 may include another inflatable chamber 42 disposed on catheter stem 14. Inflatable chamber 42 shares a common seal 44 with its neighboring inflatable chambers 12. Seals 44 fluidly seal the inflatable chambers 12 and 42 to catheter stem 14. Inflatable chamber 42 may be expanded by fluid passing through inflation lumen 16 via an inflation port 46. When inflated, inflatable chamber 42 may flare out deformable side aperture 34 for placing a branch stent (not shown) at the bifurcation.

The guide wire 4 can be provided with an X-ray-opaque coating such that its position within the vessel can be monitored. Alternatively, the guide wire may take the form of an expansive balloon.

Reference is now made to FIG. 4, which illustrates a catheter 50, constructed and operative in accordance with another embodiment of the invention. Catheter 50 is similar to catheter 40, with like elements being designated by like numerals.

Catheter 50 may include a plurality (two shown) of inflatable chambers 52 disposed on catheter stem 14. One side of inflatable chamber 52 shares a common seal 54 with its neighboring inflatable chamber 12, while the other side is an underfold 56 sealed to catheter stem 14. When inflated, inflatable chamber 52 may flare out deformable side aperture 34 for placing a branch stent (not shown) at the bifurcation.

It is appreciated that various features of the invention which are, for clarity, described in the contexts of separate embodiments, may also be provided in combination in a single embodiment. Conversely, various features of the invention which are, for brevity, described in the context of a single embodiment, may also be provided separately or in any suitable subcombination.

What is claimed is:

1. A catheter comprising:
at least one inflatable chamber disposed on a catheter stem and in fluid communication with at least one inflation lumen in said catheter stem, said at least one inflatable chamber comprising an expandable covering with an underfold fluidly sealed to said catheter stem, said underfold comprising a portion of the expandable covering that is folded underneath itself.

2. The catheter according to claim 1, comprising at least two of said inflatable chambers disposed longitudinally along said catheter stem.

3. The catheter according to claim 2, wherein said inflatable chambers abut one another in an inflated state.

4. The catheter according to claim 2, wherein said catheter stem comprises at least one lateral exit disposed between said at least two inflatable chambers.

5. The catheter according to claim 1, further comprising another inflatable chamber disposed on said catheter stem.

6. The catheter according to claim 5, wherein said other inflatable chamber shares a common seal with said at least one inflatable chamber, said seal fluidly sealing them to said catheter stem.

7. The catheter according to claim 4, further comprising a first guide wire disposed within said catheter stem.

8. The catheter according to claim 4, further comprising a second guide wire adapted to exit the catheter stem through said at least one lateral exit.

9. The catheter according to claim 1, further comprising a deformable stent positioned coaxially around the inflatable chambers and adapted to radially deform upon application of an expansive force.

10. The catheter according to claim 9, wherein said deformable stent comprises a deformable side aperture.
